(19) United States
(12) Patent Application Publication
(10) Pub. No.: US 2016/0089550 A1
(43) Pub. Date: Mar. 31, 2016

(54) TREATMENT SYSTEMS, METHODS, AND APPARATUSES FOR ALTERING THE APPEARANCE OF SKIN

(71) Applicant: Zeltiq Aesthetics, Inc., Pleasanton, CA (US)

(72) Inventors: Leonard C. DeBenedictis, Dublin, CA (US); George Frangineas, JR., Fremont, CA (US)

(21) Appl. No.: 14/855,017

(22) Filed: Sep. 15, 2015

Related U.S. Application Data
(60) Provisional application No. 62/055,572, filed on Sep. 25, 2014.

(51) Int. Cl. A61N 7/02

(52) U.S. Cl. CPC A61N 7/02; A61N 2007/0034

(57) ABSTRACT

Systems and methods that enable tissue cooling and delivery of energy for altering tissue are described herein. Aspects of the disclosure are directed to, for example, heating tissue at treatment zones to ablate, denature, reorganize, coagulate, or otherwise alter the tissue to affect the cosmetic appearance of the patient. The method can include, for example, removing heat from the target region of the human subject before, during, and/or after energy delivery to cool tissue to a temperature below normal body temperature to inhibit pain.

### CROSS-REFERENCE TO RELATED APPLICATION

The present application claims the benefit of U.S. Provisional Patent Application No. 62/055,572, filed Sep. 25, 2014, which is incorporated herein by reference in its entirety.

### INCORPORATION BY REFERENCE OF COMMONLY-OWNED APPLICATIONS AND PATENTS

The following commonly assigned U.S. Patent Applications and U.S. Patents are incorporated herein by reference in their entirety:

U.S. Patent Publication No. 2008/0287839 entitled “METHOD OF ENHANCED REMOVAL OF HEAT FROM SUBCUTANEOUS LIPID-RICH CELLS AND TREATMENT APPARATUS HAVING AN ACTUATOR”;

U.S. Pat. No. 6,032,675 entitled “FREEZING METHOD FOR CONTROLLED REMOVAL OF FATTY TISSUE BY LIPOSUCTION”;

U.S. Patent Publication No. 2007/0255362 entitled “CRYOPROTECTANT FOR USE WITH A TREATMENT DEVICE FOR IMPROVED COOLING OF SUBCUTANEOUS LIPID-RICH CELLS”;

U.S. Pat. No. 7,854,754 entitled “COOLING DEVICE FOR REMOVING HEAT FROM SUBCUTANEOUS LIPID-RICH CELLS”;

U.S. Pat. No. 8,337,539 entitled “COOLING DEVICE FOR REMOVING HEAT FROM SUBCUTANEOUS LIPID-RICH CELLS”;

U.S. Patent Publication No. 2008/0077201 entitled “COOLING DEVICES WITH FLEXIBLE SENSORS”;

U.S. Patent Publication No. 2008/0077211 entitled “COOLING DEVICE HAVING A PLURALITY OF CONTROLLABLE COOLING ELEMENTS TO PROVIDE A PREDETERMINED COOLING PROFILE”;

U.S. Patent Publication No. 2009/0118722, filed Oct. 31, 2007, entitled “METHOD AND APPARATUS FOR COOLING SUBCUTANEOUS LIPID-RICH CELLS OR TISSUE”;

U.S. Patent Publication No. 2009/0018624 entitled “LIMITING USE OF DISPOSABLE SYSTEM PATIENT PROTECTION DEVICES”;

U.S. Pat. No. 8,823,927 entitled “SYSTEM FOR TREATING LIPID-RICH REGIONS”;

U.S. Patent Publication No. 2009/0018625 entitled “MANAGING SYSTEM TEMPERATURE TO REMOVE HEAT FROM LIPID-RICH REGIONS”;

U.S. Patent Publication No. 2009/0018627 entitled “SECURE SYSTEM FOR REMOVING HEAT FROM LIPID-RICH REGIONS”;

U.S. Patent Publication No. 2009/0018626 entitled “USER INTERFACES FOR A SYSTEM THAT REMOVES HEAT FROM LIPID-RICH REGIONS”;

U.S. Pat. No. 6,041,787 entitled “USE OF CRYOPROTECTIVE AGENT COMPOUNDS DURING CRYOSURGERY”;

U.S. Pat. No. 8,285,390 entitled “MONITORING THE COOLING OF SUBCUTANEOUS LIPID-RICH CELLS, SUCH AS THE COOLING OF ADIPOSE TISSUE”;

U.S. Provisional Patent Application Ser. No. 60/941,567 entitled “METHODS, APPARATUSES AND SYSTEMS FOR COOLING THE SKIN AND SUBCUTANEOUS TISSUE”;

U.S. Pat. No. 8,275,442 entitled “TREATMENT PLANNING SYSTEMS AND METHODS FOR BODY CONTOURING APPLICATIONS”;

U.S. patent application Ser. No. 12/275,002 entitled “APPARATUS WITH HYDROPHILIC RESERVOIRS FOR COOLING SUBCUTANEOUS LIPID-RICH CELLS”;

U.S. patent application Ser. No. 12/275,014 entitled “APPARATUS WITH HYDROPHOBIC FILTERS FOR REMOVING HEAT FROM SUBCUTANEOUS LIPID-RICH CELLS”;

U.S. Pat. No. 8,603,073 entitled “SYSTEMS AND METHODS WITH INTERRUPT/RESUME CAPABILITIES FOR COOLING SUBCUTANEOUS LIPID-RICH CELLS”;

U.S. Pat. No. 8,192,474 entitled “TISSUE TREATMENT METHODS”;

U.S. Pat. No. 8,702,774 entitled “DEVICE, SYSTEM AND METHOD FOR REMOVING HEAT FROM SUBCUTANEOUS LIPID-RICH CELLS”;

U.S. Pat. No. 8,676,338 entitled “COMBINED MODALITY TREATMENT SYSTEMS, METHODS AND APPARATUS FOR BODY CONTOURING APPLICATIONS”;

U.S. Patent Publication No. 2011/0238050 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Patent Publication No. 2011/0238051 entitled “HOME-USE APPLICATORS FOR NON-INVASIVELY REMOVING HEAT FROM SUBCUTANEOUS LIPID-RICH CELLS VIA PHASE CHANGE COOLANTS, AND ASSOCIATED DEVICES, SYSTEMS AND METHODS”;

U.S. Patent Publication No. 2012/0239123 entitled “DEVICES, APPLICATION SYSTEMS AND METHODS WITH LOCALIZED HEAT FLUX ZONES FOR REMOVING HEAT FROM SUBCUTANEOUS LIPID-RICH CELLS”;

U.S. Patent Publication No. 2014/0277219 entitled “MULTI-MODALITY TREATMENT SYSTEMS, METHODS AND APPARATUS FOR ALTERING SUBCUTANEOUS LIPID-RICH TISSUE”; and

U.S. Patent Publication No. 2014/0277302 entitled “TREATMENT SYSTEMS WITH FLUID MIXING SYSTEMS AND FLUID-COOLED APPLICATORS AND METHODS OF USING THE SAME”.

### TECHNICAL FIELD

The present disclosure relates generally to treatment devices, systems, and methods for affecting the appearance of a patient. In particular, several embodiments are directed to treatment systems and methods for providing an analgesic effect and delivering energy to improve the appearance of a patient.

### BACKGROUND

Wrinkles can affect the appearance of skin on the face, as well as other areas of the body, and may be an indicator of age. For example, wrinkles are often present around the eyes, mouth, forehead, neck, etc. Exposure to ultraviolet radiation and tobacco smoke can accelerate the skin's aging process and result in premature wrinkling. As the skin naturally ages, cell division reduces, skin loosens, and skin sags. Age-related wrinkling of the skin can be promoted and/or exacerbated by habitual facial expressions or sleeping patterns, as well as poor hydration. Wrinkles, loose or sagging skin, and other skin abnormalities are often considered cosmetically unappealing and are often treated using invasive or non-invasive cosmetic procedures. Invasive cosmetic procedures often involve cutting, removing, and/or paralyzing tissue in order to lift tissue (e.g., tissue around the eyes), tighten skin, or otherwise improve the appearance of tissue. Invasive procedures, however, tend to be associated with high costs, discomfort (e.g., postoperative pain that may last for hours, days, or weeks), long recovery times, and/or risk of complications (e.g., infection). Conventional non-invasive procedures often involve delivering energy (e.g., radiofrequency energy, ultrasound energy, etc.) to ablate, coagulate, reorganize, or denature volumes of epidermal, dermal, or subcutaneous tissues which results in tissue tightening and other beneficial cosmetic effects. One drawback of these non-invasive procedures is that the patient may experience significant amounts of pain during and/or after these procedures, some of which can be relatively long.

### BRIEF DESCRIPTION OF THE DRAWINGS

In the drawings, identical reference numbers identify similar elements or acts.

FIG. 1 is a partially schematic, isometric view of a treatment system for non-invasively delivering energy to a subject's tissue in accordance with an embodiment of the disclosure.

FIG. 2 illustrates tissue and an applicator delivering energy for heating tissue at treatment zones in accordance with an embodiment of the disclosure.

FIG. 3 illustrates the heated treatment zones in accordance with an embodiment of the disclosure.

FIG. 4 is a side view of an applicator in accordance with an embodiment of the disclosure.

FIG. 5 is a cross-sectional view of a connector taken along line 5-5 of FIG. 4.

FIG. 6 is a schematic cross-sectional view of a portion of the applicator taken along line 6-6 of FIG. 4.

FIGS. 7-11 are bottom views of applicator heads in accordance with embodiments of the disclosure.

FIGS. 12-16 illustrate various stages for treating a subject in accordance with embodiments of the disclosure.

FIG. 17 illustrates treatment zones near a patient's eye in accordance with embodiments of the disclosure.

FIG. 18 is a schematic block diagram illustrating computing system software modules and subcomponents of a computing device suitable to be used in the treatment system of FIG. 1 in accordance with an embodiment of the technology.

### DETAILED DESCRIPTION

### A. Overview

The present disclosure describes treatment systems and methods for improving the appearance of a subject. Several of the details set forth below are provided to describe the following examples and methods in a manner sufficient to enable a person skilled in the relevant art to practice, make and use them. Several of the details and advantages described below, however, may not be necessary to practice certain examples and methods of the technology. Additionally, the technology may include other examples and methods that are within the scope of the technology but are not described in detail.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, stages, or characteristics may be combined in any suitable manner in one or more examples of the technology. The headings provided herein are for convenience only and are not intended to limit or interpret the scope or meaning of the technology.

At least some embodiments can be directed to reducing wrinkles, tightening loose/sagging skin, and treating other skin conditions often considered cosmetically unappealing. A treatment system can improve the skin's appearance by delivering energy to treatment zones to cause tightening of tissue to reduce the number of visible wrinkles and/or reduce the size of wrinkles (e.g., depth, length, etc.). In one embodiment, tissue can be ablated, coagulated, or thermally altered to tighten and thereby lift tissue, such as brow tissue, cheek tissue, etc. The treatment system can noninvasively cool the subject's tissue before, during, and/or after energy delivery to enhance patient comfort. In some procedures, targeted tissue can be cooled to a temperature equal to or lower than an analgesic temperature at which nerve tissue is at least partially numbed to block temperature-induced pain signals from being perceived by the brain. Additionally, the treatment system can control the temperature of the targeted tissue to prevent or control tissue freezing to prevent unwanted freezing injury. For example, the cooled tissue can be kept at or above its freezing point to avoid, minimize, or limit permanent freeze damage. In some embodiments, tissue can be controllably frozen to achieve a desired effect. A freeze event can be detected and the tissue can remain frozen for a predetermined period of time after the freeze event. The period of time can be less than, for example, 5, 10, 15, 20, 25, 30, 35, 40, 45, 50, 55, or 60 seconds or another suitable time period to prevent excess damage to the skin. As such, soft freezing can affect tissue without destroying the tissue. For example, soft freezing can improve the appearance of the skin (e.g., reduce or eliminate wrinkles), tighten skin, and/or rejuvenate skin, and if the freezing period is controlled and kept sufficiently short, adverse tissue effects can be avoided.

In some embodiments, a method for affecting tissue of a subject with skin includes applying an applicator to an external surface of the subject's skin. The applicator can cool the external surface to an extent sufficient to provide a numbing effect to bulk tissue. In one procedure, the bulk tissue can be cooled to a bulk temperature equal to or lower than about −10° C., −5° C., 0° C., 5° C., or 10° C. After the numbing effect temperature is reached in the bulk tissue, the applicator can sequentially deliver energy to discrete volumes of tissue within the bulk tissue such that treatment zones are sequentially heated to at least about 50° C., 55° C., 60° C., 65° C., 70° C., 75° C., or 80° C. while the cold-induced numbing effect inhibits pain from the heat (e.g., pain in the bulk tissue and/or in the heated treatment zones). In one embodiment, the treatment zones are microscopic zones within the dermal layer and spaced apart from the external surface. After heating the tissue, the heated tissue will be passively cooled over a very short period of time (on the order of a few hundred milliseconds) to a comfortable temperature (e.g., a temperature less than about 43° C., 45° C., etc.) by the thermal mass of the bulk tissue, which is at the numbing effect temperature. The applicator can be moved back and forth along the subject's skin to treat a targeted region.

In one embodiment, a treatment system can have an applicator configured to be applied to a subject's face to treat facial tissue, such as tissue around the eyes, mouth, forehead, etc. The applicator can cool the tissue to produce an analgesic effect without thermally damaging the tissue. After producing the analgesic effect, the applicator can non-invasively deliver energy to create one or more treatment zones within the cooled region of tissue to alter (e.g., ablate, reorganize or remodel connective tissue, denature, coagulate, etc.) tissue within such treatment zones. The analgesic effect can minimize, limit, or substantially prevent pain felt by the patient during the heating process or portion thereof.

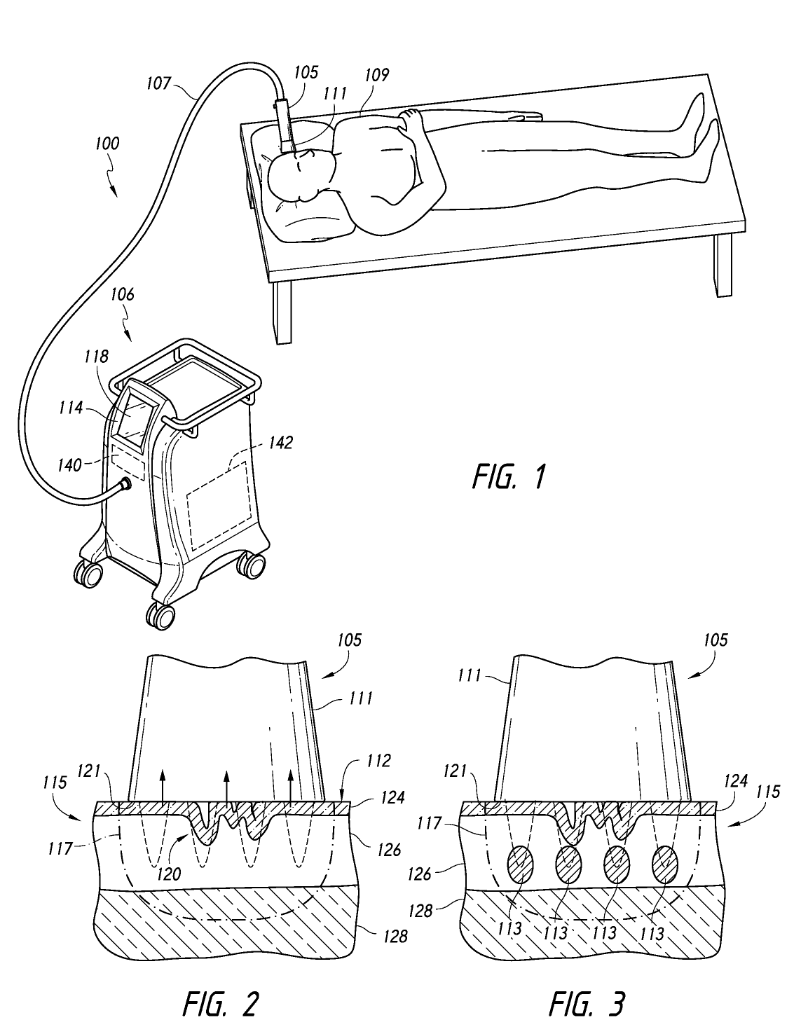

FIG. 1 is a partially schematic, isometric view of a treatment system 100 for non-invasively treating a patient in accordance with an embodiment of the disclosure. The treatment system 100 is suitable for altering one or more physiological parameters of a human subject's dermal tissue, or in other embodiments, sub-dermal tissue layers. The term “dermal tissue” means the layer of dense connective tissue that supports the epidermis 124 (FIG. 2) or outermost surface epithelium of the subject. Below the dermal tissue 126 (FIG. 2) lies the sub-dermal tissue layers 128 (FIG. 2), including the subcutaneous fat, or adipose tissue, which is composed primarily of lipid-rich cells, or adipocytes. Together, the layers of the skin 115 (FIG. 2) have a thickness of approximately 2 mm-3 mm and comprise the epidermis 124 and dermis 126. The epidermis 124 is about 100 μm thick wherein the outermost layer, the stratum corneum (15-20 μm thick) is a dense, keratinized tissue that provides penetration resistance and water-resistance. The dermal tissue layer 126 is composed of nerves, fats, blood vessels, elastin and collagen fibers which provide the skin's elasticity and smooth surface.

Disclosed herein are several embodiments of methods directed to treatment of a human subject's dermal tissue or subcutaneous tissue using one or more energy-delivery treatment modalities that can improve one or more physiological parameters corresponding to skin or body appearance in a manner that prevents pain or substantially reduces the sequelae of pain or discomfort associated with the energy delivery treatment, thereby providing for localized treatment regimens. In some embodiments, the treatment system 100 and methods described herein are suitable for altering the appearance of skin at or around the face and neck of the subject. In other embodiments, the targeted skin area can include the back, chest, arms, legs, buttocks, etc. Various physiological parameters can be affected to have an improved physical appearance, such as reduction of wrinkles, reduction of fine lines, increased skin tightness, etc. Accordingly, in various embodiments, methods described herein can be performed on the subject to improve one or more physiological parameters associated with aging and/or injury to the skin or improve body appearance.

In several embodiments, delivery of energy via an energy-delivery treatment modality (e.g., focused ultrasound, pulsed radio-frequency, focused laser, etc.) in the dermal or subcutaneous tissue can improve one or more physiological parameters, such as promoting collagen production, decreasing sebum production, and/or increasing elasticity through remodeling of connective tissue or reducing fat. However, such energy delivery and resultant heating of tissue can result in perceived pain by the subject. The subject can perceive pain as a result of pain receptor stimulation of dermal and sub-dermal sensory nerve receptors (i.e., nociceptors) or pain receptors in the sub-dermal layers. Further, pain can be the result of reactive edema and of release of cytokines and chemo-reactive agents present in the dermal tissue as a result of the treatment.

Somatic pain is perceived upon signal firing of nociceptive, touch, and pressure receptors in the dermal, sub-dermal and/or musculoskeletal tissues and is transmitted to the spinal cord and finally to the brain centers. In the dermal tissue, somatic pain may present as a burning or prickling pain and/or otherwise be characterized as a sharp pain. Acute somatic pain, e.g., nociception, can occur with the instant onset of a painful sensation in response to stimulation of the dermal and sub-dermal sensory nerve receptors (e.g., via energy delivery, heating of tissue to effect change in the dermis, etc.). Following treatment-induced injury via energy-delivery (e.g., heating of dermal tissue), inflammation can also cause pain that can be more persistent and last, for example, hours or days.

In one embodiment, methods and systems for improving one or more physiological parameters corresponding to skin or body appearance in a manner that prevents pain or reduces the sequelae of pain or discomfort associated with the energy delivery treatment include cooling the external surface of the subject's skin to provide an analgesic (e.g., numbing) effect to dermal tissue within the skin or target tissue in other tissue layers. Without being bound by theory, cooling of the dermal tissue can create a conduction block in dermal and sub-dermal sensory nerve fibers innervating these affected tissues, thereby providing an analgesic effect. A decrease in nerve conduction correlates with a decrease in temperature with myelinated fibers being the first affected nerve fibers. Evans P. J., Lloyd J. W., Jack T. M. Cryoanalgesia for intractable perineal pain. J. R. Soc. Med. 1981; 74:804-809. Cooling of the targeted tissue may also prevent and/or reduce pain associated with muscle spasm of the treated area, thus reducing the effects of ischemia secondary to the treatment-induced trauma. Hocutt J. E. Cryotherapy. Am. fam. Phys. 1981; 23:141-144. Accordingly, aspects of the present technology are directed to systems and methods for affecting dermal tissue of the subject via delivery of energy to heat treatment zones in the dermal tissue (e.g., to at least about 50° C., to at least about 60° C., etc.) after cooling the external surface of the subject's skin or target tissue to provide a numbing/analgesic effect prior to heating, thereby improving comfort during and/or after treatment.

In addition to the blocking or reduction of nerve conduction of dermal and sub-dermal sensory nerve fibers for prevention and/or reduction of acute somatic pain perception, local cold exposure may also reduce post-treatment tissue swelling and inflammation and thereby reduce pain associated with a treatment-induced inflammatory response. For example, cooling has been shown to reduce cellular enzymatic activity that can allow cells to survive with low oxygen supply in treated tissue. Increasing the cellular survival rate can reduce the volume of cellular debris and curb a physiological inflammatory response within the affected area. McLean D. The use of cold and superficial heat in the treatment of soft tissue injuries. Br. J. Sports Med. 1989. 23: 53-54.

Without being bound by theory, the selective effect of cooling the external surface of the skin to provide a numbing effect to dermal tissue within the skin or subcutaneous tissue below the skin and sequentially delivering energy to heat treatment zones within the dermal tissue and/or subcutaneous tissue is believed to result in, for example, enhanced appearance of skin (or body contour) associated with the treatment zones while inhibiting or reducing pain experienced by the subject. For example, one or more physiological properties (e.g., presence and/or number of wrinkles and fine lines, skin firmness, skin tightness, etc.) may be improved in a manner that prevents pain or reduces the sequelae of pain or discomfort associated with the energy delivery treatment. For example, when cooling the dermal tissue to a temperature lower than 10° C., conduction within sensory nerve fibers present within or adjacent to the treatment zones may be blocked or reduced. In various embodiments, non-invasive or minimally invasive cooling of the dermal tissue within or adjacent to the treatment zones can be used without harming the overlying epidermal skin or dermal tissue.

As discussed above, cooling of the dermal tissue layer of skin can provide an analgesic effect, and, as such, thermal conduction can be used to cool the desired layers of skin and/or deeper tissue to a temperature at or above the freezing point of the tissue so as to avoid an associated risk of freezing the upper layers of skin. Without being bound by theory, it is believed that uncontrolled low temperatures may potentially cause damage in the epidermis and/or dermis via at least intracellular and/or extracellular ice formation. The ice may expand and rupture the cell wall, but it may also form sharp crystals that locally pierce the cell wall as well as vital internal organelles, either or both resulting in cell death. When extracellular water freezes to form ice, the remaining extracellular fluid becomes progressively more concentrated with solutes. The high solute concentration of the extracellular fluid may cause intracellular fluid to be driven through the semi-permeable cellular wall by osmosis, resulting in cell dehydration and death.

In one typical procedure, an applicator or a cooling element is positioned at least proximate to the surface of a subject's skin and heat is removed from the underlying dermal tissue through the upper epidermal layers of the skin. Protection of the overlying cells (e.g., typically water-rich cells) from freeze damage during dermatological and related aesthetic procedures that require sustained exposure to cold temperatures may include improving the freeze tolerance and/or freeze avoidance of these skin cells (e.g., through the use of cryoprotectants or other freezing point depressant formulations by application of such formulations on the skin surface).

In some procedures, a treatment system can controllably freeze tissue to elicit a desired effect without causing significant cold-induced tissue damage. In one embodiment, a system for treating tissue includes a first element for cooling a surface of a patient's skin, a second element for detection of a freeze event in the patient's skin, and an electronic control unit for controlling the first element to continue cooling the patient's skin after the freeze event is detected to continue freezing the skin for a predetermined period of time, which is controlled to be sufficiently short so that significant adverse effects are avoided. In some embodiments, the predetermined period of time can be less than 5, 10, 15, 20, 25, 30, 35, 40, 45, 50, 55, or 60 seconds. For example, the predetermined period of time can be within a range of about 1 to 60 seconds. The freezing event can cause fibrosis of the dermis and/or epidermis, increase a protein synthesis rate of one or more of the proteins (e.g., shock proteins), and/or produce a healing response that affects the appearance of skin.

In other procedures, the treatment system can be configured to rapidly change tissue temperatures within and/or adjacent to the treatment zones over large temperature ranges. For example, an energy delivery pulse in a treatment zone, configured to result in a rapid temperature increase within the treatment zone, can be followed by cooling (e.g., via thermal diffusion, etc.) to quickly bring the temperature of the tissue within the treatment zone to the bulk cooling temperature of the surrounding tissues. Without being bound by theory, it is believed that a rapid, large temperature swing of the tissue on either the upside (temperature increase) and downside (temperature decrease), or both, within the treatment zones causes shock-induced damage to the affected tissue that can improve the appearance of the skin (e.g., reduce or eliminate wrinkles), tighten skin, and/or rejuvenate skin. In some embodiments, the energy delivery steps can heat the tissue within the treatment zones to a heated temperature that is lower than a temperature used to coagulate tissue or lower than conventional heating therapies that cannot take advantage of the large temperature swings produced by one or more embodiments of the present invention. In these arrangements, the temperature shock-induced damage caused by the large and/or rapid fluctuations in temperature can induce the desired improvements to the skin.

In some embodiments, the treatment system 100 can cool the skin of the patient to a temperature in a range of from about −20° C. to about 20° C. In other embodiments, the cooling temperatures can be from about −15° C. to about 10° C., from about −5° C. to about 10° C., from about 0° C. to about 10° C., or from about 0° C. to about 5° C. In further embodiments, the cooling temperatures can be equal to or lower than 10° C., or in yet another embodiment, from about −20° C. to about 10° C. The desired cooled temperature can, in some embodiments, be less than −2° C., less than 0° C., less than 3° C., less than 4° C., or less than 5° C.

In certain embodiments, the skin can be cooled rapidly (e.g., within about 20-35 seconds, within about 30-60 seconds, etc.) so as to minimize discomfort experienced by the subject associated with creating the cooling temperatures. In some embodiments, skin can be cooled from an initial temperature to the desired cool temperature in less than 100 seconds, less than 90 seconds, less than 80 seconds, less than 70 seconds, less than 60 seconds, less than 50 seconds, less than 40 seconds, less than 30 seconds, etc. For example, a cool down rate can be equal to or greater than about 0.5° C./second (e.g., 1° C./second, 2° C./second, 3° C./second, etc.). In some embodiments, the cooldown rate selected can depend on the starting temperature of the subject's skin and the heat removal capacity of the elements used to cool (e.g., thermoelectric coolers (TECs)). For example, for skin having an initial temperature of about 30° C., a cooling procedure can include cooling the skin using TECs, which can create an initial cooling rate of about 2° C./second. As the skin cools to temperatures approaching about 0° C. to about −10° C., the cooling rate can decrease from the first cooling rate to a second cooling rate of about 1° C./second. In some embodiments, the system can provide variable-rate cooling to cool the bulk tissue at a generally constant rate or according to a patient-specific temperature profile.

### B. Treatment Systems

FIG. 1 shows the treatment system 100 including a handheld facial applicator 105 (“applicator 105”), a console unit 106, and a connector 107 extending between the applicator 105 and console unit 106. The applicator 105 can include a temperature-controlled cooling element or head 111 (“head 111”) for conductively cooling a treatment site and for delivering energy to targeted tissue. The treatment site can be cooled to a sufficient extent to produce an analgesic effect to inhibit pain before, during, and/or after energy delivery to, for example, enhance patient comfort (e.g., induce post treatment skin desensitization), allow a relatively high treatment speed with high density treatment zones, and/or reduce swelling and/or inflammation. The applicator 105 can be continuously or intermittently moved along a human subject 109 while continuously or intermittently delivering energy to ablate, denature, or otherwise affect the targeted tissue.

The applicator 105 can pre-cool treatment zones and bulk tissue to an analgesic temperature. The applicator can then deliver energy to raise the temperature of microscopic zones of target tissue to a temperature sufficient to elicit a response for removing or reducing undesired features, including skin irregularities. Skin irregularities may include wrinkles characterized by folds, ridges, or creases in the skin that often are the result of an aging process and promoted by sun damage, smoking, habitual facial expression, skin dehydration, and other factors. The delivered energy can be acoustic energy (e.g., focused ultrasound energy) that heats tissue within small microscopic treatment zones to ablate, denature, reorganize, remodel, coagulate, or otherwise affect the tissue to, for example, reduce wrinkles, tighten tissue (e.g., sagging or loose skin), or lift tissue. The density, pattern, and/or rate of creation of treatment zones can be selected such that the tissue within the treatment zones quickly returns to a temperature at or near the bulk temperature of the bulk tissue after cessation of energy delivery to such treatment zones due to the large thermal capacity of the bulk tissue, so that temperature-induced pain is either totally absent or substantially reduced. The applicator 105 can continue to cool the bulk tissue around the heated tissue at the treatment zones to further inhibit pain by, for example, desensitizing tissue to enhance post-treatment comfort, inhibit swelling, inhibit inflammation, or combinations thereof.

The treatment system 100 can monitor the treatment site using closed-loop control to determine when tissue is ready to be treated (e.g., after cooling has achieved a temperature sufficiently low to induce a desired numbing effect). The treatment system 100 can then comfortably deliver the energy to target tissue at the numbed treatment site. The treatment can be continuously or periodically monitored to ensure that the bulk tissue is maintained at or below a numbing temperature (e.g., 10° C.). The treatment system 100 can also monitor the treatment site to avoid unwanted events, such as permanent cold-induced injury. For example, the tissue can be kept at or below the numbing temperature to maintain the numbing effect and also at or above a freezing point of the tissue to avoid permanent freeze damage.

FIG. 2 is a schematic cross-sectional view of facial tissue and the applicator 105 delivering energy in the form of focused ultrasound energy in accordance with an embodiment of the disclosure. FIG. 3 illustrates heated treatment zones in the tissue in accordance with an embodiment of the disclosure. Referring now to FIG. 2, the applicator 105 has been applied to an external surface 112 of the subject's skin 115 with wrinkles 120. The applicator 105 can include the head 111 with a temperature-controlled surface 121 for absorbing heat (represented by arrows) to produce an analgesic effect in a volume of bulk tissue 117 that may include, for example, the epidermis 124, dermis 126, and/or hypodermis 128. In some procedures, the bulk dermal tissue 117 can be cooled to a temperature low enough to anesthetize the tissue at the treatment zones before energy delivery and optionally after energy delivery.

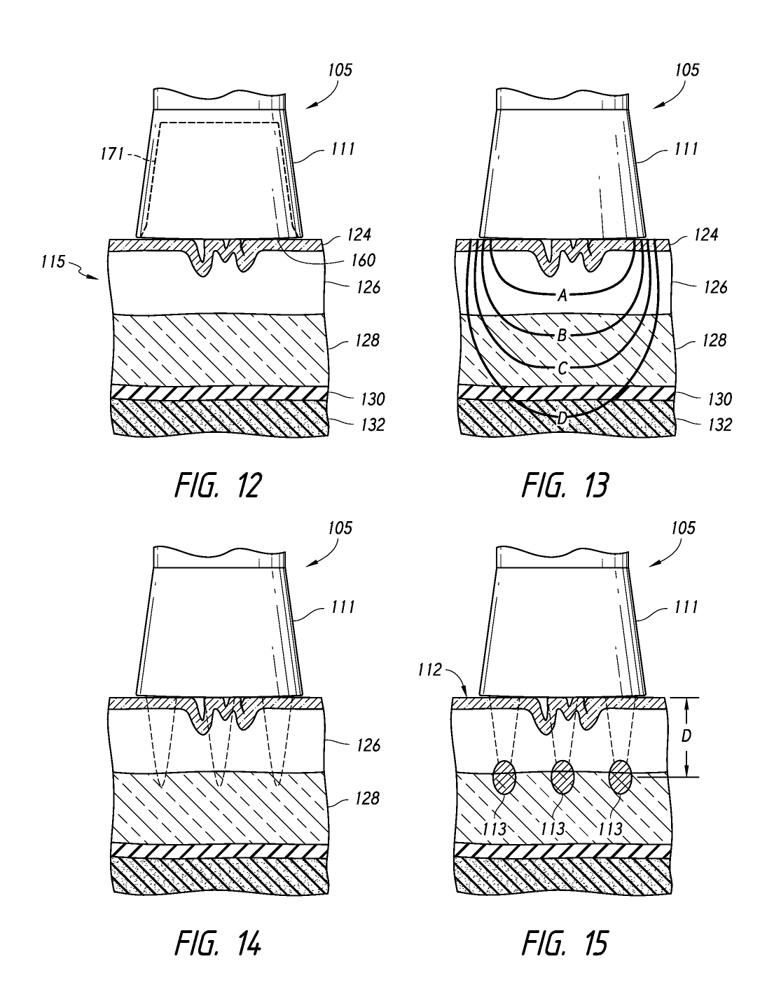

The applicator 105 can deliver energy (illustrated in dashed line) to create one or more microscopic treatment zones (four zones are illustrated in FIG. 2) while the analgesic effect inhibits sensation in the heated tissue or adjacent tissue. The energy can rapidly heat the tissue at treatment zones (e.g., treatment zones 113 in FIG. 3) to a target temperature within a short period of time, and since volumes of the heated tissue are microscopic in size they will each be rapidly cooled back down to a temperature of the bulk tissue to help minimize or limit the pain, if any, experienced by the user. The locations, density, and/or a rate of creation of the treatment zones can be controlled such that the microscopic treatment zones quickly return to a desired pre-cooled analgesic bulk temperature after cessation of energy delivery to such treatment zone. The applicator 105 can be passed repeatedly back and forth along the patient's skin to achieve a desired density of treatment zones. Exemplary methods are discussed in connection with FIGS. 12-16.

Referring again to FIG. 1, the console unit 106 can provide energy and coolant to the applicator 105 and can include an electronic control unit in the form of a controller 114, an energy-generating unit or drive unit 140 (“drive unit 140”), and a thermal assembly 142. The thermal assembly 142 can hold and cool liquid coolant that is continuously or intermittently delivered to the applicator 105 via the connector 107. The coolant can circulate through the applicator 105 to absorb heat, and the heated coolant can flow from the applicator 105 back to the console unit 106 via the connector 107.

The drive unit 140 can provide power for the applicator 105 and can include an ultrasound signal generator configured to supply a drive signal to one or more transducers of the applicator 105. In other embodiments, the drive unit 140 can be configured to provide energy or signals to the applicator 105 for outputting radiofrequency energy, microwave energy, light energy, mechanical energy (e.g., vibrations, massage energy, etc.), and/or electromagnetic energy. To deliver radiofrequency energy, the drive unit 140 can be a radiofrequency (RF) electrical generator, and the applicator 105 can include one or more RF electrodes.

The thermal assembly 142 can include, without limitation, one or more refrigeration units, cooling towers, thermoelectric chillers, or other devices capable of removing heat from or adding heat to coolant. In one embodiment, the thermal assembly 142 includes one or more reservoirs for holding liquid, thermal units for cooling the liquid, and pumps for pumping the liquid through the system. The reservoirs can be, without limitation, one or more tanks or other suitable containers with a sufficient fluid holding capacity. The thermal units can include, without limitation, one more refrigeration units that operate based on a vapor-compression refrigeration cycle or other refrigeration cycle. Additionally or alternatively, the thermal units can include one or more thermoelectric devices, such as Peltier devices. The pumps can be positive displacement pumps (e.g., reciprocating pumps, gear pumps, rotary vane pumps, etc.), rotodynamic pumps, or other types of drive devices capable of pressurizing coolant such that the coolant flows through the connector 107 and the applicator 105.

The controller 114 can exchange data with the applicator 105 via an electrical line or, alternatively, via a wireless or an optical communication link to monitor and/or control the treatment. The controller 114 can include any processor, Programmable Logic Controller, Distributed Control System, secure processor, and the like. A secure processor can be implemented as an integrated circuit with access-controlled physical interfaces; tamper resistant containment; means of detecting and responding to physical tampering; secure storage; and shielded execution of computer-executable instructions. Some secure processors also provide cryptographic accelerator circuitry. Secure storage may also be implemented as a secure flash memory, secure serial EEPROM, secure field programmable gate array, or secure application-specific integrated circuit. The controller 114 can include an input/output device 118 (shown as a touch screen) that functions as both an input device and an output device. In alternative examples, the controller 114 may be part of the applicator 105.

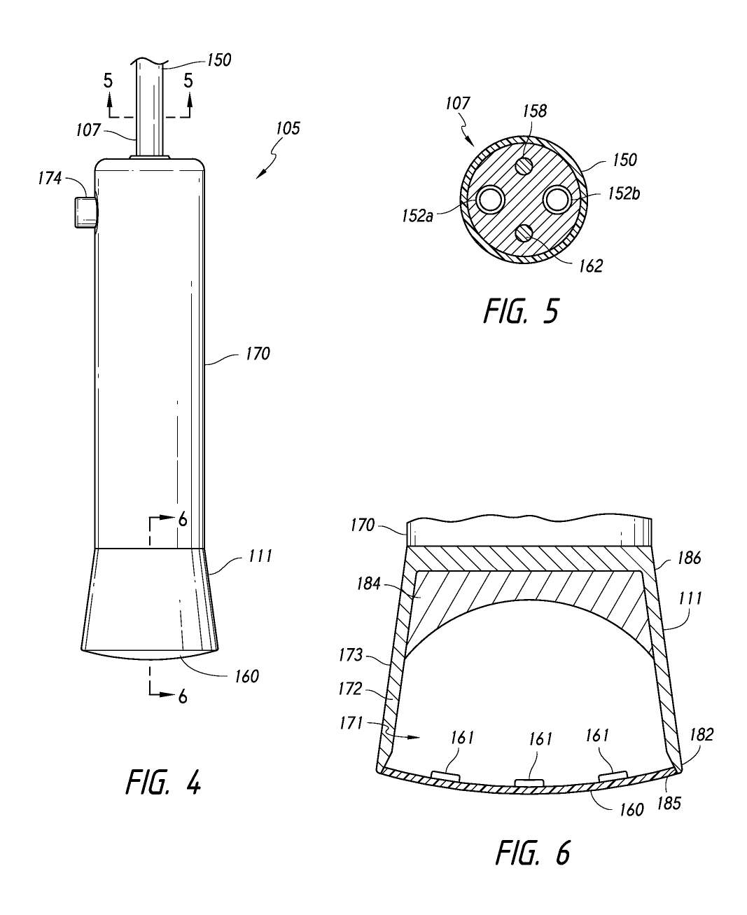

FIG. 4 is a side view of the applicator 105 in accordance with an embodiment of the disclosure. FIG. 5 is a cross-sectional view of the connector 107 taken along line 5-5 of FIG. 4. Referring now to FIG. 5, the connector 107 can include a main body 150 (e.g., a solid or hollow main body), a supply fluid line or lumen 152a (“supply fluid line 152a”), and a return fluid line or lumen 152b (“return fluid line 152b”). The main body 150 may be configured (via one or more adjustable joints) to “set” in place for the treatment of the subject. Chilled coolant from the thermal assembly 142 (FIG. 1) flows through the supply fluid line 152a to the applicator 105, and coolant from the applicator 105 flows through the return fluid line 152b. The supply and return fluid lines 152a, 152b can be tubes made of polyethylene, polyvinyl chloride, polyurethane, and/or other materials that can accommodate circulating coolant, such as water, glycol, propylene glycol, ethylene glycol, synthetic heat transfer fluid, oil, refrigerant, and/or any other suitable heat conducting fluid.

The connector 107 can also include one or more lines 158 for providing output (e.g., drive signals, energy, power, etc.) to the applicator 105 (FIG. 1) and one or more control lines 162 for providing communication between the console unit 106 (FIG. 1) and the applicator 105 (FIG. 1). The line 158 and control line 162 (and other lines including, but not limited to fluid lines 152a, 152b) may be bundled into or otherwise accompanied by a conduit or the like to protect such lines, enhance ergonomic comfort, minimize unwanted motion (and thus potential inefficient removal of heat and/or delivery of energy), and to provide an aesthetic appearance. Examples of such a conduit include a flexible polymeric, fabric, or composite sheath, an adjustable arm, etc. and may be designed (via adjustable joints, etc.) to “set” the conduit in place for the treatment of the subject 109.

Referring again to FIG. 4, the applicator 105 can include a main body 170 and the head 111. FIG. 6 is a detailed cross-sectional view of a portion of the applicator taken along line 6-6 of FIG. 4. Referring now to FIG. 4, the main body 170 can include one or more fluid lines, electrical lines, power lines, circuitry, or other components for connecting features of the connector 107 with features of the head 111. A control element 174 (e.g., a button, a switch, a dial, etc.) positioned along the main body 170 can be used to adjust, for example, operation of the applicator 105. Control panels, input devices (e.g., touch pads, keypads, touch screens, etc.) and/or output devices (e.g., lights, screens, etc.) may be contained in, attached to, or integrated with the applicator 105. In some embodiments, the applicator 105 is fluid cooled as discussed in connection with FIG. 6. In certain embodiments, the applicator 105 can include both thermoelectric elements and fluid cooling features (e.g., fluid chambers, an array of cooling channels, etc.). In non-fluid cooled embodiments, the applicator 105 can include one or more thermoelectric devices capable of heat/cooling tissue without utilizing coolant.

FIG. 6 shows an embodiment of the fluid cooled head 111 connected to the main body 170. The head 111 can include a head body 173, a chamber 171, an energy emitter in the form of a transducer 184, and a contact element 160. The head body 173 can include an insulated sidewall 172 for thermally insulating the chamber 171, and the sidewall 172 can have a lower portion 182 connected to a periphery 185 of the contact element 160 and an upper portion 186 carrying the transducer 184. The chamber 171 can be a sealed chamber for holding circulating coolant such that substantially no coolant escapes during treatment. The transducer 184 can be configured to output ultrasound energy that travels through the coolant filled chamber 171, through the contact element 160, and into tissue underneath the contact element 160.

The contact element 160 can include, without limitation, one or more membranes made, in whole or in part, of plastic, rubber, silicon, or other compliant material through which energy can travel. The dimensions (e.g., thickness, surface area, etc.) and properties (e.g., thermal conductivity) of such membranes can be selected based on, for example, desired mechanical properties, thermal properties, and energy transmitting capabilities. The contact element 160 can be compliant to conform to a wide range of highly contoured regions. In other embodiments, the contact element 160 can be generally rigid (e.g., a metal plate with a high thermal conductivity) for applying pressure to tissue. Other contact elements with other constructions and comprising other materials can be used. The thermal properties of the contact element 160 can be selected based on the target cooling rates and characteristics of the energy to be delivered.

The transducer 184 can be configured to emit ultrasound energy that can be focused, unfocused, or defocused for achieving a desired therapeutic effect. The applicator 105 can have circuitry and/or one or more lenses or other components for controlling and/or focusing ultrasound delivery. In one embodiment, the transducer 184 has an array (e.g., a linear array) of addressable transducer elements or a phased array of transducer elements to provide focusing capability. FIG. 6 shows the transducer 184 having a curved configuration for focusing the ultrasound energy to a focal zone. Transducer elements of the transducer 184 can generate ultrasonic energy from electrical energy (e.g., electrical energy from the drive unit 140 of FIG. 1) and can be, for example, piezoelectric elements. The piezoelectric elements can comprise a piezoelectrically active material, such as piezoelectric ceramic, composite material, or other suitable active material. Piezoelectric ceramics can comprise a crystalline material (e.g., quartz) that changes shape in response to an applied electrical current. This change in shape, made oscillatory by an oscillating driving signal, creates ultrasonic sound waves that can be transmitted via, for example, the coolant, contact element 160, etc. Other types of transducer elements can be used.

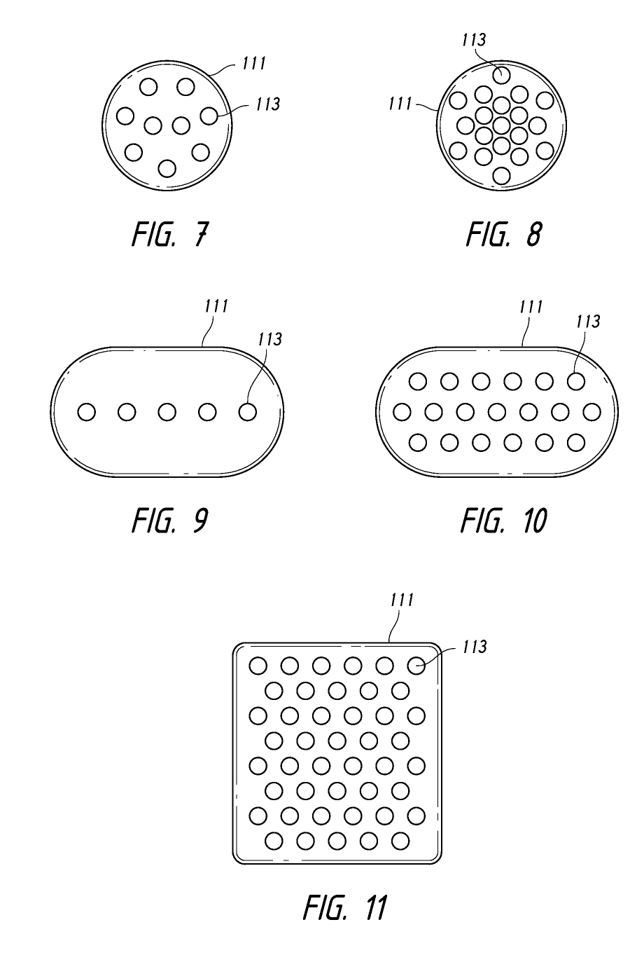

FIGS. 7-11 are bottom views of various embodiments of the applicator head 111. FIG. 7 shows the head 111 with a generally circular shape and configured to define an array of fractional or treatments zones 113 (one identified) that are generally evenly spaced apart. The pattern of the treatment zones can be selected to minimize treatment zone overlap or compensate for treatment zone overlap. For example, the pattern density at the central region of the head 111 of FIG. 8 can be significantly greater than the pattern density towards the periphery of the head 111 to compensate for treatment zone overlap when using radial scanning techniques. FIG. 9 shows the head 111 suitable for linear scanning techniques and configured to define a linear array of treatment zones 113 (one identified). FIG. 10 shows the head 111 configured to define an array of evenly spaced apart treatment zones 113 (one identified) that are evenly spaced apart. FIG. 11 shows the head 111 having a generally polygonal shape (e.g., rounded square shape) and configured to define a square-shaped pattern of treatment zones 113 (one identified). The configuration of the transducer 184 and head 111 can be selected based on desired characteristics of the treatment zone pattern (e.g., patterns with uniform or variable spacing, regular patterns, irregular patterns, etc.), such as number of treatment zones, density of treatment zones, etc.

The system discussed herein can be used with a set of applicators, each of which can be designed to treat identified portions of the patient's body (e.g., face, neck, chin, cheeks, head, chest, and so forth). The surface area of the head 111 (FIG. 1) for treating facial tissue can be in a range of about 5 cm² to about 20 cm². In certain embodiments, the surface area is about 8 cm² to about 15 cm². Applicator heads for treating large areas (e.g., chest, stomach, etc.) can have a relatively large surface area, for example an area in a range of about 30 cm² to about 70 cm², about 40 cm² to about 60 cm², etc. These large area applicators can be configured to heat treatment zones comprising tissue of the epidermis, dermal tissue, connective tissue, superficial muscular aponeurotic tissue, adipose tissue, and/or muscle tissue.

### C. Suitable Treatment Methods

FIGS. 12-16 illustrate various stages for treating a site using the treatment system 100 of FIG. 1 in accordance with embodiments of the disclosure. The method generally includes applying an applicator to an external surface of the subject and using the applicator to reduce the temperature of the subject's skin (e.g., remove heat from underlying tissue layers) to anesthetize and/or numb the tissue. After an analgesic temperature is reached, the applicator can also noninvasively deliver energy to heat tissue at microscopic treatment zones within the subject's skin to affect the tissue at the zones (e.g., affecting tissue to improve one or more physiological parameters corresponding to skin appearance). In various arrangements, pain experienced by the subject and associated with or caused by the delivery of the energy to the treatment zones can be prevented, reduced, minimized, or otherwise controlled by the analgesic (e.g., numbing) effect to the affected tissue provided by the cooling. This process can be repeated any number of times to treat a region of the subject. Even though the procedure is described below with reference to the treatment system 100 of FIG. 1, the method may also be applied in other treatment systems with additional or different features.

The user can select a treatment program using the controller 114 (FIG. 1), which can store algorithms used to, for example, determine timing of energy delivery, durations of energy delivery, characteristics of the energy, and other treatment parameters. The controller 114 can receive input (e.g., instructions, data, etc.) from an input/output device 118 (illustrated as a touch screen) and/or exchange data with another computing device (e.g., smartphone, computer, etc.). In other embodiments, the console unit 106 (FIG. 1) can include keyboard, a mouse, a stylus, a touch screen, a push button, a switch, a potentiometer, a scanner, an audio component such as a microphone, a printer, a video monitor, a medium reader, an audio device such as a speaker, any combination thereof, and any other device or devices suitable for accepting user input and/or display and/or for providing user feedback.

FIG. 12 shows the head 111 placed against the subject's skin 115. A substance can be applied to the subject to couple the head 111 to the skin 115. For example, the substance can be an ultrasound transmitting gel that facilitates ultrasound delivery to tissue while allowing the movement of the applicator head 111 along the patient's skin. The ultrasound transmitting gel can be a cryoprotectant gel (e.g., a cryoprotectant gel described in commonly assigned U.S. Patent Publication Nos. 2007/0255362 and 2014/0005760) that can be applied topically to lower the freezing point of the tissue to minimize, limit, or substantially prevent permanent freeze damage to such tissue. Other substances can be used to facilitate energy delivery to the treatment site. In other embodiments, the head 111 can be applied directly to the subject without using any coupling substance. In some procedures, a single gel can serve both functions of coupling acoustic energy into the skin and lowering a freezing point of the skin.

When the applicator 105 is positioned at the treatment site, chilled coolant can circulate through the chamber 171 (shown in phantom line) to cool the contact element 160, which in turn conductively cools the tissue. The tissue at the treatment site can be cooled to create temporary or reversible conduction blocks in dermal and sub-dermal sensory nerve fibers innervating these affected tissues so as to provide the analgesic effect. In one procedure, the dermal tissue can be rapidly numbed in less than about 10 seconds, 20 seconds, 30 seconds, 40 seconds, 50 seconds, 60 seconds, 70 seconds, 80 seconds, 90 seconds, 100 seconds, or other desired cooling period. The coolant can be at a temperature within a range of about −20° Celsius to about 10° Celsius, about −15° Celsius to about 5° Celsius, or about −5° Celsius to about 5° Celsius, or other suitable temperature range for achieving desired cooling rates. In some embodiments, cooling rates of the skin surface or targeted tissue can be equal to or greater than about 0.2° C./second, 0.5° C./second, 1° C./second, 1.5° C./second, 2° C./second, 3° C./second, or other desired cooling rates selected based on, for example, patient preference.

FIG. 13 illustrates a cross-sectional temperature profile produced by the applicator 105. Isothermal curves show the temperatures that are reached at different depths due to the cooling. By way of example, it is possible to achieve temperatures in which isotherm A=−15° C. to 0° C., B=−5° C. to 5° C., C=0° C. to 10° C., and D=5° C. to 15° C. In one procedure, the isotherm A=−10° Celsius, B=0° Celsius, C=5° Celsius, and D=10° Celsius. Accordingly, the tissue above the isotherm D can be at least partially numb due to the cooling. Thus, treatment zones can be painlessly created in the epidermis 124, dermis 126, subcutaneous tissue 128, superficial muscular aponeurotic tissue 130, and/or muscle 132 using a wide range of energy delivery rates. To create treatment zones located within the dermis 126, cooling can produce isotherms A and B (e.g., A=5° Celsius and B=10° Celsius) such that bulk tissue above the isotherm B is numbed. Other temperature distributions can be produced by controlling operation of the treatment system 100.

The treatment system 100 can monitor the treatment site to avoid a freezing event in the tissue in the skin or underlying tissue and, in some embodiments, it detects and responds to freezing events by, for example, stopping cooling, changing from a cooling mode to a heating mode, and/or alerting the user to apply substance(s), such as cryoprotectant. Methods and systems for collecting feedback data and monitoring of temperature measurements and detecting freeze events are described in commonly assigned U.S. Pat. No. 8,285,390, which is incorporated by reference in its entirety. In some embodiments, the controller 114 (FIG. 1) can be programmed to cause the applicator 105 to reduce the temperature of the bulk dermal tissue at or near the treatment zones from a normal temperature to a cooled temperature to anesthetize the bulk dermal tissue. The controller 114 can also be programmed to cause the applicator 105 to deliver energy to the treatment zones to increase the temperature of the treatment zones to at least about 50° C. while maintaining the cooled temperature at or above a temperature that causes permanent cold-induced tissue injury. In some procedures, the applicator 105 can perform a soft freeze procedure to controllably freeze the skin 117. The applicator 105 can cool the treatment site to compensate for heat of fusion due to the freezing event. The sensors 161 (FIG. 6) can be used to detect the freeze event. Other types of sensors can be used to detect freeze events, cold-induced injury, etc.

FIG. 14 shows the head 111 delivering focused ultrasound energy (illustrated in dashed line) at focal zones sufficiently deep to inhibit or prevent damage to the surface of the skin. In some procedures, the tissue is heated to a target peak temperature (e.g., 50° C., 60° C., 70° C., 80° C., etc.) within a short period of time (e.g., less than 50 milliseconds, less than 100 milliseconds, less than 200 milliseconds, less than 300 milliseconds, less than 400 milliseconds, less than 500 milliseconds, etc.). The applicator 105 can continue to cool the bulk tissue surrounding the treatment zones to help maintain the analgesic effect while successive microscopic treatment zones under the head 111 are heated.

The ultrasound applicator 105 of FIG. 14 has focal zones near the interface between the dermis 126 and the subcutaneous tissue 128 and is suitable for use on the dermal facial tissue or relatively shallow tissue. In other embodiments, the applicator 105 can be configured to deliver energy at deeper focal zones comprising connective tissue, adipose tissue, and/or muscle. In other embodiments, the energy can be delivered to more shallow levels, such as the mid or upper dermis, and including the dermal tissue. Accordingly, depths of the focal zones can be selected based on the depths of the tissue being targeted and the clinical effect being sought. For example, the applicator 105 can be configured to heat tissue at a depth D (i.e., the distance from the skin surface 112 to the center of the treatment zones 113 in FIG. 15) equal to or less than 2 mm, 3 mm, 4 mm, 5 mm, 6 mm, or 7 mm. In some embodiments, the transducers are configured to heat tissue at a depth between about 3 mm and 5 mm, more than 4.5 mm, more than 6 mm, and anywhere in the ranges of 0-3 mm, 0-4.5 mm, 0-25 mm, 0-100 mm, or other desired depths. Thus, the depth D can be about 1 mm to about 2 mm for facial procedures, about 4 mm to about 6 mm for deeper fat/connective tissue procedures, or other desired depths selected based on the targeted tissue. In some embodiments, the applicator 105 can provide acoustic power in range of about 1 W to about 50 W and at a frequency of about 0.1 MHz to about 15 MHz. It can be pulsed with the pulse duration less than, for example, 50 milliseconds, 100 milliseconds, 200 milliseconds, or 300 milliseconds and can emit about 1,000-10,000 W/cm², 1,000-8,000 W/cm², or 1,000-7,000 W/cm² to reach a tissue temperature equal to or greater than about 50° C., 60° C., 70° C., 80° C., or other desired temperature. Other treatment settings for the applicator 105 can be used.

FIG. 15 shows treatment zones 113 that can be ellipsoid-shaped, prolate spheroid-shaped (e.g., football-shaped), spherical, or the like. Microscopic spherical-shaped zones can have diameters equal to or less than about 8 mm, 7 mm, 6 mm, 5 mm, 4 mm, 3 mm, 2 mm, 1 mm, or 0.5 mm. In one embodiment, the diameters of microscopic spherical treatment zones 113 can be in a range about 1 mm to about 1.5 mm. Microscopic prolate spheroid-shaped treatment zones 113 can have a diameter equal to or less than about 8 mm, 7 mm, 6 mm, 5 mm, 4 mm, 3 mm, 2 mm, 1.5 mm, 1 mm, or 0.5 mm. In one embodiment, prolate spheroid-shaped treatment zones 113 can have diameters along their minor axis in a range of about 1 mm to about 1.5 mm and an aspect ratio equal to about 3:1, about 2:1, or desired aspect ratio. The shape, depth, and configuration of the treatment zones can be selected based on tissue characteristics and procedure to be performed.

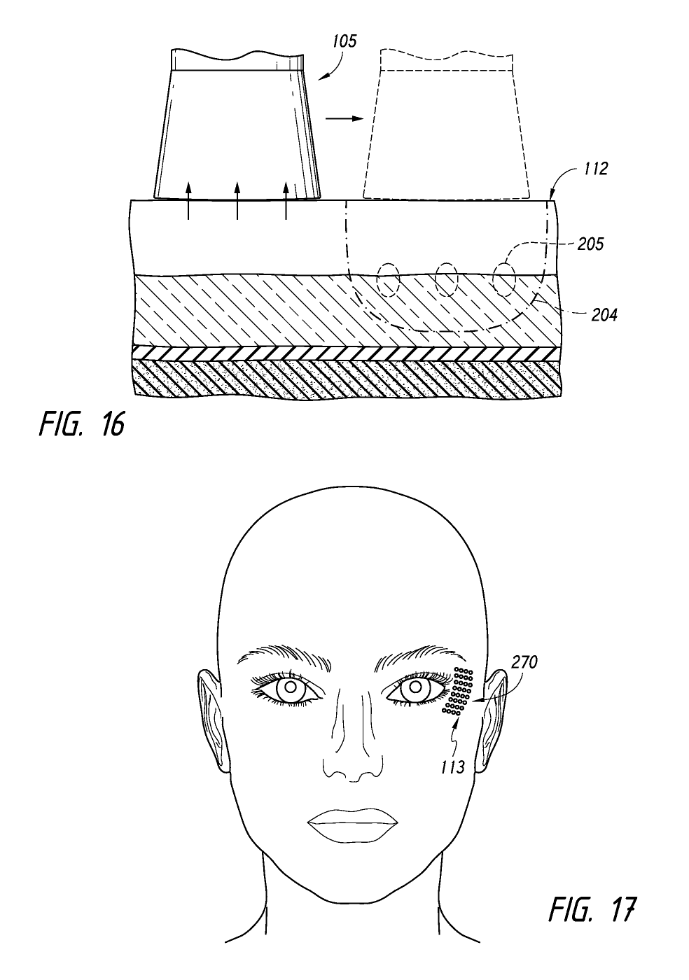

FIG. 16 shows the applicator 105 after stopping the energy delivery shown in FIG. 15. The heated microscopic tissue can be cooled to a comfortable, non-pain-inducing temperature (e.g., about 43° C. to about 45° C.) by the bulk tissue within a relatively short period of time defined by the thermal time constants of the bulk tissue and microscopic treatment zones (e.g., several hundred milliseconds) to avoid or minimize heat-induced pain.

The applicator 105 can continue to absorb heat as it is slid along the surface 112 to another treatment position (illustrated in phantom line). The applicator 105 can cool a region of tissue 204 and can then heat tissue at treatment zones 205 (one identified). This process can be repeated any number of times to create tens, hundreds, and/or thousands of treatment zones.

FIG. 17 illustrates treatment zones 113 near a patient's eye with a relatively dense pattern created using a single pass. To produce a more dense pattern of treatment zones, the applicator 105 can be passed multiple times across treatment site 270 to create new treatment zones with each pass. Thus, the number of passes can be chosen based on the desired treatment zone density. In some embodiments, the applicator 105 can have a density per pass of about 20%. The applicator can be passed two or more times across the treatment site to produce a density of about 30% to 40% (e.g., for two passes), depending on the amount of treatment zone overlap. The total density can be progressively increased by increasing the number of passes.

The treatment system 100 can also controllably produce a cold-induced response that improves the appearance of skin, tightens skin, rejuvenates skin, reduces or eliminates skin wrinkles, reduces or enhances the appearance of cellulite, and/or treats sebaceous glands. Before, during, and/or after energy delivery, a soft freezing process can be used to improve the appearance of the dermis while limiting injury to the skin. In some soft freeze procedures, skin tissue can be controllably frozen to cause fibrosis in the dermis and/or epidermis, increase a protein synthesis rate of one or more proteins (e.g., shock proteins), and/or produce a healing response that affects the dermis. In one embodiment, the applicator 105 can cool the external surface of the patient's skin while monitoring the tissue. For example, the sensors 161 (FIG. 6) can detect one or more freeze events in the cooled tissue. The controller 114 (FIG. 1) can control operation of the applicator 105 to keep at least a portion of the dermis in its frozen state for a predetermined period of time after the freeze event is detected. The predetermined period of time can be selected to achieve a desired response without causing excessive damage to the skin. In one procedure, the predetermined period of time can be in a range of about 5, 10, 15, 20, 25, 30, 35, 40, 45, 50, 55, or 60 seconds. Other periods of time can be selected.

In additional embodiments, the treatment system 100 can controllably produce temperature shock-induced damage to the tissue in the treatment zones so as to improve the appearance of skin (e.g., tighten skin, rejuvenate skin, reduce or eliminate skin wrinkles, reduce or enhance the appearance of cellulite, and/or treat sebaceous glands). Temperature shock-induced changes to the treated tissue can be induced via a rapid and/or large temperature shift within the tissue. For example, cellular injury due to temperature shock can result in increased protein synthesis of one or more proteins (e.g., shock proteins) and/or produce a healing response that affects the treated tissue. In one embodiment, the applicator 105 can rapidly heat tissue within the treatment zones with delivered energy pulse(s). Once the tissue is rapidly heated, the applicator 105 can continue to cool the external surface of the patient's skin to maintain a cooled bulk temperature while monitoring the tissue temperature. The heated tissue zones, each of which is microscopic in size, will cool rapidly via thermal diffusion to the temperature of the bulk tissue surrounding/adjacent to the heated tissue regions. The sensors 161 (FIG. 6) can be used to detect the temperature of the bulk tissue, and optionally the temperature peaks and troughs of the microscopic treatment zones. The controller 114 can control the operation of the applicator 105 to heat tissue to a threshold upper temperature and to detect cooling of the tissue (e.g., via thermal diffusion) and/or actively remove heat from the treated tissue. Predetermined rates of heating and cooling can be selected to achieve a desired response without causing excessive damage to the skin and can be selected based on the desired mechanism of action and response. In some embodiments, the controller 114 can deliver energy to heat the tissue within the treatment zones to a heated temperature that is lower than a temperature used to coagulate tissue. In these arrangements, temperature shock-induced damage caused by the large and/or rapid temperature fluctuations can induce the desired improvements to the skin.

Although specific examples are described herein, one skilled in the art will be capable of identifying other methods that could be performed. Moreover, the methods described herein can be altered in various ways. The controller 114 (FIG. 1) can store treatment plans designed to reduce/eliminate skin irregularities, sagging skin, loose skin, etc. For example, one segment can be performed to reduce wrinkles (e.g., age-related wrinkles), and another segment can be performed at the same or different treatment site to tighten loose tissue. The controller 114 can cause the applicator 105 to cycle through each segment of a treatment plan. Using temperature sensors (e.g., the sensors 161 in FIG. 6) proximate to the patient's skin, the controller 114 can evaluate, for example, the skin temperature and/or whether a temperature or heat flux is sufficiently close to the target temperature or heat flux. Although a region of the body (e.g., epidermis, dermis, subcutaneous tissue, adipose tissue, etc.) may have been cooled or heated to the target temperature, in actuality that region may be close to but not at the target temperature, e.g., because of the body's natural heating and cooling variations. Thus, although the system may attempt to heat or cool tissue to the target temperature(s) or to provide a target heat flux, a sensor may measure a sufficiently close temperature or heat flux. Additional sensors may be included for measuring tissue impedance, treatment application force, tissue contact with the applicator, and energy interaction with the skin of the subject, among other process parameters. For example, applicators (e.g., applicator 105 of FIG. 1) can have pressure sensors configured to monitor pressure applied to the subject's face and thereby maintain a desired level of comfort.

The treatment system 100 can be a multi-modality system configured to alter a human subject's subcutaneous adipose tissue, including such as by cooling and/or by delivering energy to subcutaneous adipose tissue. For example, the treatment system 100 can deliver ultrasound energy tissue (e.g., dermal tissue) and also cool other tissue (e.g., subcutaneous tissue). For example, the treatment system 100 can perform methods for non-invasively reducing tissue as disclosed in U.S. Pat. No. 7,367,341 entitled “METHODS AND DEVICES FOR SELECTIVE DISRUPTION OF FATTY TISSUE BY CONTROLLED COOLING” to Anderson et al. and U.S. Patent Publication No. 2005/0251120 entitled “METHODS AND DEVICES FOR DETECTION AND CONTROL OF SELECTIVE DISRUPTION OF FATTY TISSUE BY CONTROLLED COOLING” to Anderson et al., the entire disclosures of which are incorporated herein by reference. The applicator 105 can be used to cool subcutaneous adipose tissue a sufficient amount to inhibit, destroy, or reduce the lipid-rich cells. Without being bound by theory, the selective effect of cooling on lipid-rich cells is believed to result in, for example, membrane disruption, shrinkage, disabling, destroying, removing, killing, or another method of lipid-rich cell alteration. Depending on the duration and final cooling temperature, subcutaneous lipid-rich cells can selectively become altered in a manner that makes the cells more susceptible to further interrogation and/or cell injury than non-lipid-rich cells in the same region. Such alteration (e.g., by cooling, energy delivery and/or combination of cooling and energy delivery) is believed to be an intermediate and/or final result of one or more mechanisms acting alone or in combination. It is thought that such mechanism or mechanisms can trigger an apoptotic cascade, which is believed to be the dominant form of lipid-rich cell death by non-invasive cooling alone or in combination with other forms of cell interrogation. Temperature exposures and/or other energy delivery modalities that elicit these apoptotic events in lipid-rich cells may contribute to long-lasting and/or permanent reduction and reshaping of subcutaneous adipose tissue.

### D. Suitable Computing Environments

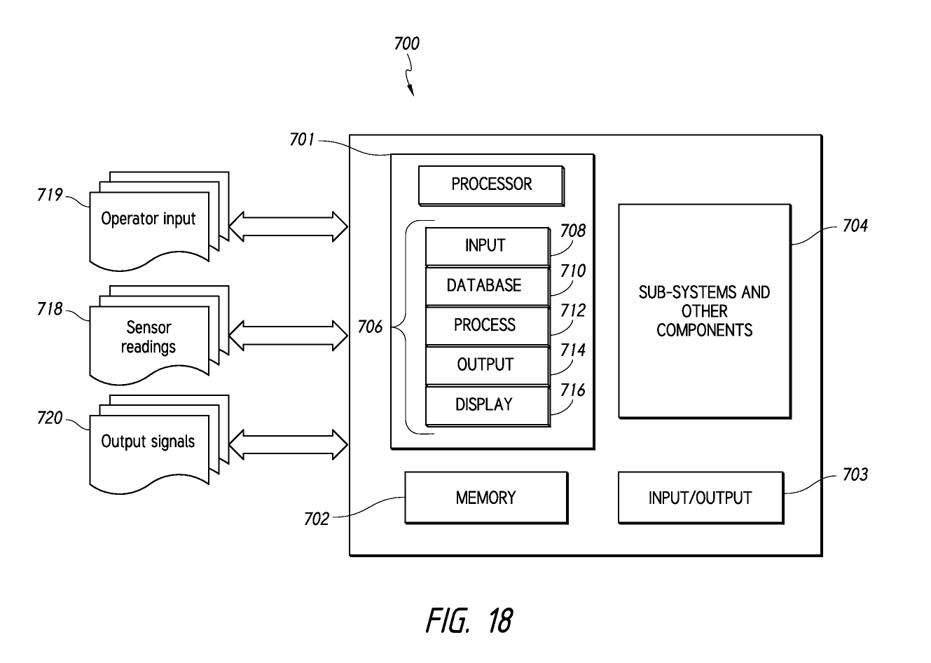

FIG. 18 is a schematic block diagram illustrating subcomponents of a controller in the form of a computing device 700 suitable for the controller 114 of FIG. 1 in accordance with an embodiment of the disclosure. The computing device 700 can include a processor 701, a memory 702 (e.g., SRAM, DRAM, flash, or other memory devices), input/output devices 703, and/or subsystems and other components 704. The computing device 700 can perform any of a wide variety of computing processing, storage, sensing, imaging, and/or other functions. Components of the computing device 700 may be housed in a single unit or distributed over multiple, interconnected units (e.g., though a communications network). The components of the computing device 700 can accordingly include local and/or remote memory storage devices and any of a wide variety of computer-readable media.

As illustrated in FIG. 18, the processor 701 can include a plurality of functional modules 706, such as software modules, for execution by the processor 701. The various implementations of source code (i.e., in a conventional programming language) can be stored on a computer-readable storage medium or can be embodied on a transmission medium in a carrier wave. The modules 706 of the processor can include an input module 708, a database module 710, a process module 712, an output module 714, and, optionally, a display module 716.

In operation, the input module 708 accepts an operator input 719 via the one or more input devices described above with respect to FIG. 1, and communicates the accepted information or selections to other components for further processing. The operator input 719 can be the location of the treatment site and desired procedure to be performed (e.g., reduction of scar tissue, tightening of tissue, etc.). The database module 710 organizes records, including patient records, treatment data sets, treatment profiles and operating records, and other operator activities, and facilitates storing and retrieving of these records to and from a data storage device (e.g., the memory 702, an external database, etc.). Any type of database organization can be utilized, including a flat file system, hierarchical database, relational database, distributed database, etc.

In the illustrated example, the process module 712 can generate control variables based on sensor readings 718 from sensors (e.g., sensors 161 of FIG. 6) and/or other data sources, and the output module 714 can communicate operator input to external computing devices and control variables to the controller 114 (FIG. 1). The display module 716 can be configured to convert and transmit processing parameters, sensor readings 718, output signals 720, input data, treatment profiles, and prescribed operational parameters through one or more connected display devices, such as a display screen, printer, speaker system, etc. A suitable display module 716 may include a video driver that enables the controller 114 to display the sensor readings 718 or other status of treatment progression on the screen.

In various embodiments, the processor 701 can be a standard central processing unit or a secure processor. Secure processors can be special-purpose processors (e.g., reduced instruction set processor) that can withstand sophisticated attacks that attempt to extract data or programming logic. The secure processors may not have debugging pins that enable an external debugger to monitor the secure processor's execution or registers. In other embodiments, the system may employ a secure field programmable gate array, a smartcard, or other secure devices.

The memory 702 can be standard memory, secure memory, or a combination of both memory types capable of storing executable instructions for performing the methods disclosed herein. By employing a secure processor and/or secure memory, the system can ensure that data and instructions are both highly secure and that sensitive operations such as decryption are shielded from observation. Suitable computing environments and other computing devices and user interfaces are described in commonly assigned U.S. Pat. No. 8,275,442, entitled “TREATMENT PLANNING SYSTEMS AND METHODS FOR BODY CONTOURING APPLICATIONS,” which is incorporated herein in its entirety by reference.

### E. Conclusion

Various embodiments of the technology are described above. It will be appreciated that details set forth above are provided to describe the embodiments in a manner sufficient to enable a person skilled in the relevant art to make and use the disclosed embodiments. Several of the details and advantages, however, may not be necessary to practice some embodiments. Additionally, some well-known structures or functions may not be shown or described in detail, so as to avoid unnecessarily obscuring the relevant description of the various embodiments. Although some embodiments may be within the scope of the technology, they may not be described in detail with respect to the Figures. Furthermore, features, structures, or characteristics of various embodiments may be combined in any suitable manner. Moreover, one skilled in the art will recognize that there are a number of other technologies that could be used to perform functions similar to those described above.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include, but not be limited to, systems that have A alone, B alone, C alone, A and B together, A and C together, B and C together, and/or A, B, and C together, etc.).

Some of the functional units described herein have been labeled as modules, in order to more particularly emphasize their implementation independence. For example, modules (e.g., the modules discussed in connection with FIG. 18) may be implemented in software for execution by various types of processors. An identified module of executable code may, for instance, comprise one or more physical or logical blocks of computer instructions which may, for instance, be organized as an object, procedure, or function. The identified blocks of computer instructions need not be physically located together, but may comprise disparate instructions stored in different locations that, when joined logically together, comprise the module and achieve the stated purpose for the module. A module may also be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like.

A module of executable code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations, including over different storage devices, and may exist, at least partially, merely as electronic signals on a system or network.

Any patents, applications, and other references, including any that may be listed in accompanying filing papers, are incorporated herein by reference. Aspects of the described technology can be modified, if necessary, to employ the systems, functions, and concepts of the various references described above to provide yet further embodiments. While the above description details certain embodiments and describes the best mode contemplated, no matter how detailed, various changes can be made. Implementation details may vary considerably, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated.

What is claimed is:

1. A method for affecting dermal tissue of a subject with skin, comprising:
applying an applicator to an external surface of the subject's skin;
cooling the external surface using the applicator to cool bulk dermal tissue in the skin to a bulk analgesic temperature equal to or lower than about 10° C. so as to provide a numbing effect to the bulk dermal tissue; and
sequentially delivering energy from the applicator to heat microscopic treatment zones within the bulk dermal tissue such that the microscopic treatment zones are sequentially heated to at least about 50° C. while the numbing effect inhibits pain in the bulk dermal tissue and the heated microscopic treatment zones.

2. The method of claim 1, further comprising:
continuing to cool the bulk dermal tissue as the energy is delivered; and
controlling delivery of the energy to keep a density and a rate of creation of the microscopic treatment zones sufficiently low such that each microscopic treatment zone returns to a temperature at or near the bulk analgesic temperature after cessation of energy delivery to such microscopic treatment zone.

3. The method of claim 1 wherein the microscopic treatment zones are heated to at least about 60° C.

4. The method of claim 1 wherein cooling the bulk dermal tissue to provide the numbing effect includes removing a sufficient amount of heat from the bulk dermal tissue for a sufficient length of time to anesthetize the bulk dermal tissue and the microscopic treatment zones before delivering any energy to such microscopic treatment zones.

5. The method of claim 1 wherein cooling the external surface includes cooling the bulk dermal tissue at a cooling rate equal to or greater than about 0.5° C. per second.

6. The method of claim 1 wherein cooling the bulk dermal tissue includes cooling the bulk dermal tissue to a temperature equal to or lower than about 5° C.

7. The method of claim 1 wherein sequentially delivering energy from the applicator to heat the microscopic treatment zones includes
delivering energy from the applicator positioned at a first position such that the energy is delivered to a first group of the microscopic treatment zones beneath the first position; and
delivering energy from the applicator positioned at a second position different from the first position such that the energy is delivered to a second group of the microscopic treatment zones beneath the second position.

8. The method of claim 1 wherein the energy is delivered without damaging the external surface of the subject's skin.

9. The method of claim 1, further comprising:
freezing at least a portion of the bulk dermal tissue;
detecting a freeze event in the bulk dermal tissue; and
controlling cooling of the bulk dermal tissue such that the bulk dermal tissue is frozen for a predetermined period of time after the freeze event.

10. The method of claim 9 wherein the predetermined period of time is between about 5 seconds and about 60 seconds.

11. The method of claim 1 wherein at least one microscopic treatment zone is heated and created in less than about 300 milliseconds and after it is so created it passively returns to a temperature equal to or very near the bulk analgesic temperature in less than about 500 milliseconds.

12. A method for affecting skin of a human subject's body, comprising:
cooling a volume of bulk dermal tissue at a treatment site at a cooling rate equal to or greater than about 0.5° C. per second such that a bulk temperature of the bulk dermal tissue is equal to or lower than about 10° C. and is at or below a bulk analgesic temperature; and
sequentially delivering energy to heat a plurality of microscopic treatment zones within the volume of bulk dermal tissue such that the bulk dermal tissue is at a significantly lower temperature than such heated treatment zones to inhibit pain in the dermal tissue, and after termination of energy delivery to each treatment zone, the temperature of each such microscopic treatment zone quickly returns to the bulk analgesic temperature.

13. The method of claim 12, further comprising continuing to cool the volume of bulk dermal tissue while delivering the energy to the microscopic treatment zones.

14. The method of claim 12 wherein delivering energy includes delivering a sufficient amount of energy to damage tissue in the microscopic treatment zones without damaging any significant amount of the bulk dermal tissue adjacent to each heated microscopic treatment zone.

15. The method of claim 12 wherein heating the plurality of microscopic treatment zones includes heating each microscopic treatment zone faster than each microscopic treatment zone is passively cooled to the bulk analgesic temperature after termination of the delivery of energy to such microscopic treatment zone.

16. A method for affecting a subject's skin, comprising:
cooling bulk dermal tissue of the subject to produce a numbing effect; and
after producing the numbing effect, delivering energy to one or more microscopic treatment zones within the cooled bulk dermal tissue such that dermal tissue in the one or more microscopic treatment zones is affected while the numbing effect inhibits pain in the bulk dermal tissue.

17. The method of claim 16 wherein cooling the bulk dermal tissue includes cooling the bulk dermal tissue from normal body temperature to an anesthetizing temperature within about 100 seconds.

18. The method of claim 16 wherein cooling the bulk dermal tissue includes cooling the bulk dermal tissue at an average cooling rate equal to or greater than about 0.5° C.

19. The method of claim 16 wherein cooling the bulk dermal tissue includes cooling the bulk dermal tissue from normal body temperature to a cooled temperature within a cooling period, wherein the cooled temperature is equal to or less than about 10° C., and wherein the cooling period is equal to or less than about 100 seconds.

20. The method of claim 19 wherein the cooled temperature is −2° C., −1° C., 0° C., 1° C., 2° C., 3° C., 4° C., 5° C., 6° C., 7° C., 8° C., 9° C., or 10° C.; and wherein the cooling period is about 100 seconds, 90 seconds, 80 seconds, 70 seconds, 60 seconds, 50 seconds, 40 seconds, 30 seconds, or 20 seconds.

21. The method of claim 16 wherein cooling the bulk dermal tissue includes reducing the temperature of the bulk dermal tissue to a sufficiently low temperature to block pain in the bulk dermal tissue adjacent to the microscopic treatment zones while energy is delivered to the microscopic treatment zones.

22. A method for affecting dermal tissue of a subject having skin, comprising:
applying an applicator to a treatment site located along the subject's skin; and
performing a treatment cycle within a period of time equal to or less than about 40 seconds, wherein performing the treatment cycle includes—
cooling bulk dermal tissue at the treatment site using the applicator, and
delivering energy from the applicator to microscopic treatment zones at the treatment site to heat the microscopic treatment zones to at least about 50° C. while the bulk dermal tissue is at a sufficiently low temperature due to cooling by the applicator so as to significantly block pain caused by the energy and/or heating of the microscopic treatment zones.

23. A cosmetic treatment method for affecting dermal tissue of a human subject having skin to achieve a cosmetically beneficial alteration of the skin, the method comprising:
removing heat from the skin of the human subject to cool bulk dermal tissue in the subject's skin to a bulk analgesic temperature; and
delivering energy to a treatment zone within the bulk dermal tissue to heat dermal tissue in the treatment zone to an extent sufficient to reorganize connective tissue in the treatment zone;
wherein the bulk analgesic temperature provides a reduction in pain associated with the energy delivery to the treatment zone.

24. The cosmetic treatment method of claim 23 wherein the bulk analgesic temperature is between about −15° C. and about 10° C.

25. The cosmetic treatment method of claim 23 wherein the bulk analgesic temperature is above a freezing point of the subject's skin.

26. The cosmetic treatment method of claim 25, further comprising applying a cryoprotectant to the subject's skin before removing heat from the skin to lower the freezing point of the skin.

27. The cosmetic treatment method of claim 23 wherein delivering energy to the treatment zone occurs after the bulk dermal tissue has been cooled to the bulk analgesic temperature.

28. A system for affecting a subject's skin, comprising:
an applicator configured to be applied to an external surface of the subject's skin; and
a controller programmed to cause the applicator to perform the method in one of claims 1-27.

29. A system for affecting a human subject's skin, comprising:
an energy-generating unit;
an applicator in electrical communication with the energy-generating unit and configured to non-invasively remove heat from bulk dermal tissue of the subject's skin and deliver energy to heat treatment zones within the bulk dermal tissue; and
a controller in communication with the energy-generating unit and having instructions for causing the system to:
use the applicator to reduce the temperature of the bulk dermal tissue at or near the treatment zones from a normal temperature to a cooled temperature to anesthetize the bulk dermal tissue, and
deliver the energy from the applicator to the treatment zones to increase the temperature of the treatment zones to at least about 50° C.

30. The system of claim 29 wherein the cooled temperature is above a temperature that causes permanent cold-induced freezing injury to the subject's skin.

31. The system of claim 29 wherein the controller is programmed to cause the system to detect a freeze event in the bulk dermal tissue; and
control operation of the applicator based on the freeze event such that at least a portion of the bulk dermal tissue is frozen for a predetermined period of time.

32. The system of claim 31 wherein the predetermined period of time is in a range of about 5-60 seconds.

33. The system of claim 29 wherein the energy delivered to the treatment zones is acoustic energy.

34. The system of claim 29 wherein the cooled temperature is between about −10° C. and about 10° C.

35. The system of claim 29 wherein the applicator is configured to maintain the cooled temperature of the bulk dermal tissue at a temperature at or below about 10° C. while delivering energy to the treatment zones.

36. The system of claim 29 wherein the controller further comprises instructions to monitor the cooled temperature of the bulk dermal tissue to maintain anesthetization of the bulk dermal tissue.

37. The system of claim 29, further comprising a cryoprotectant for being applied between the applicator and a surface of the subject's skin to lower the freezing point of the skin.

38. The system of claim 37 wherein the cryoprotectant further comprises an acoustic coupling gel.
